(19) United States
(12) Patent Application Publication
(10) Pub. No.: US 2012/0277156 A1
(43) Pub. Date: Nov. 1, 2012

(54) INHIBITION OF PATHOLOGICAL BONE FORMATION

(75) Inventors: Ted Gross, Seattle, WA (US); Steve Bain, Shoreline, WA (US); Sean Nork, Seattle, WA (US)

(73) Assignee: UNIVERSITY OF WASHINGTON TRHOUGH IT'S CENTER FOR COMMERCIALIZATION, SEATTLE, WA (US)

(21) Appl. No.: 13/386,043

(22) PCT Filed: Jul. 21, 2010
(86) PCT No.: PCT/US2010/042741

§ 371 Date: Mar. 9, 2012

Related U.S. Application Data
(60) Provisional application No. 61/227,168, filed on Jul. 21, 2009.

(51) Int. Cl. A61K 38/16; A61K 31/445; A61P 25/00; A61P 19/08; A61P 17/02; A61K 31/167; A61K 31/4745

(52) U.S. Cl. 514/16.7; 514/626; 514/330; 514/279

(57) ABSTRACT

Described are methods of inhibiting heterotopic ossification (HO) in a subject in need thereof. The methods involve administering an effective amount of a proprioception inhibitor to the subject, whereby HO is inhibited or prevented. The present invention also relates to a method of treating a subject with bone trauma. This involves administering a proprioception inhibitor to the subject under conditions effective to treat the bone trauma, where the proprioception inhibitor prevents or inhibits HO.

### CROSS REFERENCE TO RELATED APPLICATIONS

This application claims the benefit of priority of U.S. Provisional Application No. 61/227,168, filed Jul. 21, 2009, the contents of which are incorporated herein by reference.

### FIELD OF THE INVENTION

The present invention is directed to inhibition of pathological bone formation.

### BACKGROUND OF THE INVENTION

Heterotopic ossification (HO) is the formation of mature lamellar bone in soft tissue sites outside the skeletal periosteum. HO is a secondary complication of spinal cord injury, traumatic brain injuries, burns, fractures, muscle contusion, joint arthroplasty, amputation following trauma, lower motor neuron disorders, and hereditary disorders (Strakowski et al., “Upper Limb Musculoskeletal Pain Syndromes,” In: Buschbaker et al. editor(s). Physical Medicine and Rehabilitation. 2nd Edition. Philadelphia: WB Saunders Company, 779 (1996)). The incidence of HO ranges from 11% to 76%, depending on the population studied and the method of diagnosis (Garland et al., “Periarticular Heterotopic Ossification in Head-injured Adults. Incidence and Location,” J Bone Joint Surgery American Volume 62(7):1143-6 (1980), Sazbon et al., “Widespread Periarticular New-bone Formation in Long-term Comatose Patients,” J Bone Joint Surgery British Volume 63(1):120-5 (1981)), with the hip joint involved in 77% of patients (Orzel et al., “Heterotopic Bone Formation: Clinical, Laboratory, and Imaging Correlation,” J Nuclear Medicine 26(2):125-32 (1985)). HO may result in joint contracture and ankylosis, pain, spasticity, swelling, fever, neurovascular compression, lymphoedema, pressure ulcers, and significant disability (Garland D E., “A Clinical Perspective on Common Forms of Acquired Heterotopic Ossification,” Clinical Orthopaedics Related Research (263):13-29 (1991)), most commonly around proximal limb joints.

### SUMMARY OF THE INVENTION

A first aspect of the present invention relates to a method of inhibiting heterotopic ossification (HO) in a subject in need thereof. This method includes administering an effective amount of a proprioception inhibitor to the subject, where HO is inhibited or prevented. It is preferred that the administration is local to, or adjacent to, the area at which one wishes to prevent, inhibit or otherwise treat HO. In one aspect, the treatment methods described herein include a step of identifying a subject at risk for or in need of the prevention, inhibition or treatment of HO. For subjects at risk for or in need of such prevention, inhibition or treatment according to the methods described herein, a proprioception inhibitor or a transient paralytic agent is administered at or substantially near the site at which one wishes to prevent or lessen HO, such that HO is prevented, inhibited or reduced.

In one aspect, transient paralysis (including, e.g., inhibition of proprioception and motor function) is induced by the agent administered. In another aspect, a transient paralytic agent is administered to inhibit or prevent HO. For simplicity, the following refers to the use of proprioception inhibitors. It should be understood that unless specifically specified otherwise, the agent administered can also be a transient paralytic agent.

In various aspects, local administration of the proprioception inhibitor may be carried out intramuscularly, by implantation, or intralesionally and with a pharmaceutically-acceptable carrier. In one embodiment, a proprioception inhibitor is administered to muscle adjacent to a transcortical bone defect. In particular embodiments, the proprioception inhibitor is selected from inhibitors of small-diameter sensory fibers including, for example, long acting, locally applied anesthetics; e.g. lidocaine, bupivicaine, veratridine, saxitoxin, Clostridium botulinum toxin, type A, and other botulinum toxin preparations that inhibit proprioception or HO, e.g., in assays as described herein. Epstein-Barash et al., “Site-specific Analgesia With Sustained Release Liposomes,” PNAS 106(17):6891-6892 (2009), which is hereby incorporated by reference in its entirety, describes the design and characterization of a novel controlled release system for site-specific delivery of saxitoxin (STX) either as a sole active ingredient or in combination with dexamethasone or bupivacaine. This approach, or others like it can provide sustained release of proprioception inhibitors of use in the methods and compositions described herein.

While agents useful for inhibiting pathological bone formation as described herein tend to cause at least local paralysis or inhibition of motor function, the methods and compositions described herein do not necessarily rely upon motor function inhibition for their effect. Without wishing to be bound by theory, the proprioception inhibitory effects of such agents are believed to be instrumental in the inhibition of bone formation. Proprioception primarily involves small-diameter sensory fibers. As such, a selective inhibitor of small-diameter sensory fibers would be a preferred proprioception inhibitor for the methods and compositions described herein. A “selective” inhibitor would inhibit small-diameter sensory fibers to a greater extent than larger-diameter motor fibers at a given dose. A benefit of a selective inhibitor would be inhibition of pathological bone growth without inhibition of motor function. It should be understood, however, that while it is believed that the proprioception-inhibiting function is involved in and possibly central to the effect on bone growth, it is not at all required that the agent be selective for inhibition of small-diameter sensory fibers, as evidenced by the effects of Botulinum toxin preparations, which also inhibit motor function.

For each method, a subject in need may be selected. The method of inhibiting HO in a subject may be carried out in a mammal, in particular, in a human.

In some embodiments of the methods and compositions described herein, a proprioception inhibitor is administered in conjunction with another agent that modulates bone growth or repair. For example, bone morphogenetic protein (BMP) family members or other bone-related growth factors may be given in conjunction with the proprioception inhibitor/paralytic drug.

The approach to the prevention of HO described herein is applicable to HO arising under any circumstances. As non-limiting examples, the HO may be due to spinal cord injury, traumatic brain injuries, burns, bone trauma, fractures, muscle contusion, joint arthroplasty, amputation following trauma, lower motor neuron disorders, and hereditary disorders. Thus, each of these conditions places one at risk of developing HO and/or in need of such treatment. In one embodiment, the joint arthroplasty may be hip replacement.

A further aspect of the present invention relates to a method of treating a subject with bone trauma. This method involves administering a proprioception inhibitor to the subject under conditions effective to treat the bone trauma, where the proprioception inhibitor prevents HO.

Another aspect of the present invention relates to the use of a proprioception inhibitor for the treatment of bone trauma. In one embodiment, the proprioception inhibitor inhibits or prevents HO.

Another aspect of the present invention relates to the use of a proprioception inhibitor for the preparation of a medicament for the inhibition or prevention of HO.

Another aspect of the present invention relates to the use of a proprioception inhibitor for the preparation of a medicament for the treatment of bone trauma.

### BRIEF DESCRIPTION OF THE DRAWINGS

FIG. 1 shows a micro-CT image of the entire tibia of a mouse used in a model for transcortical defects (left) and a cross-sectional image (above), showing location and the penetrating nature of the transcortical defect.

FIG. 2 shows that serial micro-CT images along a 3 mm region of the tibial diaphysis in a saline-treated mouse (top) clearly demonstrate the exuberant periosteal osteogenic response to a uni-cortical defect both distal and proximal to the defect site (osteogenic response outlined in white). This response was similar in appearance to the intramembranous bone formation induced following bone fracture. Further, it can be seen that the defect is being repaired by calcifying tissues within the defect hole (white arrow in mid diaphyseal image). In contrast, Clostridium botulinum toxin, type A (“BT×A”) treatment of the calf inhibited osteogenesis along the entire length of the diaphysis (bottom), without affecting calcifying tissues immediately adjacent to the injury and within the injury itself.

FIG. 3 shows the mean (±SE) summed volume of periosteal new bone formation stimulated by a uni-cortical defect in saline and BT×A treated mice. A single dose of BT×A that transiently inhibited calf muscle function resulted in an 87.5% decrease in osteogenic tissue.

FIG. 4 shows that BT×A injection of the calf muscles reduced Bone Volume (BV) of the periosteal callus by 83.1% vs. saline-injected control mice but had no effect on BV of the endocortical callus. *P<0.05; n=4 mice per group.

FIG. 5. Three dimensional microCT image of the diaphyseal region from a mouse treated with BT×A injections in the calf muscles (left) compared to a microCT image from an animal that received a BT×A injection directly into the bone defect (right). Note the absence of inhibitory effects on bone healing at the defect site (dark arrow), and the presence of periosteal osteogenesis on bone surface (white arrows). Compared to the mice treated with BT×A injections of the calf muscles, the osteogenic response to bone injury was 70.5% greater (P<0.05) when BT×A was injected directly into the bone defect; n=4 mice per group.

FIG. 6 shows the results of studies of the effect of transient neuromuscular signaling blockage on trauma-induced periosteal bone formation in a surgically-induced skeletal trauma model described in Example 3. BT×A injection of muscle proximal to the tibial defect site profoundly inhibits osteogenic response to skeletal trauma.

FIG. 7 further shows a MicroCT image of the tibial defect site following transient paralysis of the quadriceps. Even though the quadriceps muscle is proximal to the defect site, inhibition of neuromuscular function inhibits periosteal osteogenic response without disturbing bone formation at the defect site.

FIG. 8 shows heterotopic ossification in the BMP-4-induced model of heterotopic ossification described herein in Example 4.

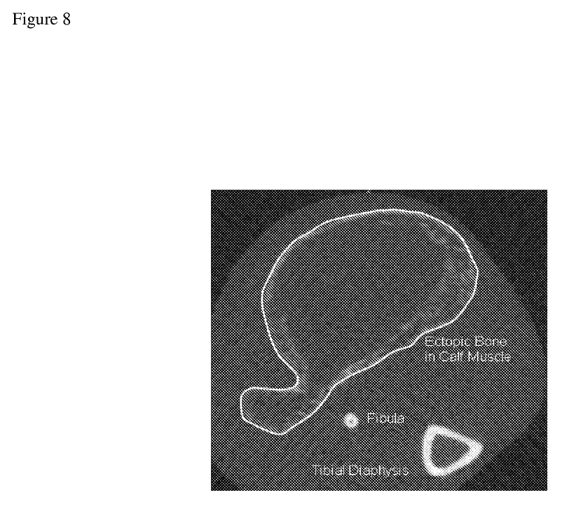

### DETAILED DESCRIPTION OF THE INVENTION

Described herein are methods and compositions for preventing or inhibiting inappropriate bone growth, including heterotopic ossification. The methods relate generally to the use of proprioception inhibitors, such as botulinum toxin, type A (“BT×A”) (e.g., BOTOX™) to inhibit or prevent inappropriate bone growth. Broadly speaking, a proprioception inhibitor is locally administered at the site where inappropriate bone growth is to be prevented or inhibited.

The following describes various aspects of the invention, including materials and things to consider in practicing the method described.

One aspect of the present invention relates to a method of inhibiting heterotopic ossification (HO) in a subject in need thereof. This method includes administering an effective amount of a proprioception inhibitor to the subject at or near the site where HO is to be prevented or inhibited, wherein HO is inhibited or prevented.

Another aspect of the present invention relates to a method of treating a subject with bone trauma in which overgrowth of bone tissue is inhibited or prevented. This method involves administering a proprioception inhibitor to the subject under conditions effective to treat the bone trauma, where the proprioception inhibitor prevents HO.

### DEFINITIONS

The term “inappropriate bone growth” relates to overgrowth of bone at the site of bone trauma beyond that necessary for healing. The term also encompasses “heterotopic ossification,” which refers more specifically to the abnormal formation of true bone within extraskeletal soft tissues.

As used herein, the term “botulinum toxin” refers to a neurotoxin produced by a Clostridium botulinum strain. Unless specifically stated, the botulinum toxin is not necessarily limited to a specific sub-type. Thus, the term encompasses sub-types A-G, to the extent that one or all of them can inhibit inappropriate bone growth or HO, e.g., in the transcortical defect model described herein.

Proprioception relates to the sensory perception of the position or arrangement of one's body or body parts in three dimensional space. A “proprioception inhibitor” interferes with or alters this sensory perception. In one aspect described herein, inhibition of proprioceptive nerves or proprioception prevents or decreases HO. Ligaments and tendons have proprioceptive nerves (mechanoreceptors and golgi tendon organs, respectively). While not wishing to exclude proprioceptive nerves that may be associated with other tissues, ligament and tendon-associated proprioceptive nerve structures may play a key role in the development of HO, especially in and around joints. Thalhammer et al., “Neurological Evaluation of a Rat During Sciatic Nerve Block With Lidocaine,” Anesthesiology 82:1013-1025 (1995), which is hereby incorporated by reference in its entirety, teaches assays for proprioception that can be used to evaluate proprioception inhibitors. The assays are described in further detail in the section titled “Proprioception Inhibitor” below and can be used to evaluate a given composition for proprioceptive inhibitory activity suitable for use in the methods and compositions described herein. A “proprioception inhibitor” as the term is used herein will result in a grade of at least 1, but potentially 2 or 3 on Thalhammer's grading scale described herein.

The neurotoxic factor botulinum toxin type A (“BT×A”) (e.g., the active ingredient in the approved formulation of Botox™) is a proprioception inhibitor as that term is used herein.

As used herein, “prevention” or “preventing,” when used in reference to a disease, disorder or symptoms thereof, refers to a reduction in the likelihood that an individual will develop a disease or disorder, e.g., heterotopic ossification. The likelihood of developing a disease or disorder is reduced, for example, when an individual having one or more risk factors for a disease or disorder either fails to develop the disorder or develops such disease or disorder at a later time or with less severity, statistically speaking, relative to a population having the same risk factors and not receiving treatment as described herein. The failure to develop symptoms of a disease, or the development of reduced (e.g., by at least 10% on a clinically accepted scale for that disease or disorder) or delayed (e.g., by days, weeks, months or years) symptoms is considered effective prevention. Regarding “inhibition” of bone growth, in certain embodiments of the invention, bone growth can be inhibited by at least about 20%, 25%, 50%, 75%, 90%, 95%, or 99% in the presence of an administered agent or composition (e.g., a botulinum toxin preparation or other proprioception inhibitor preparation) when compared to growth of bone in the absence of an agent or composition. In other embodiments of the invention, inappropriate bone growth (e.g., HO) can be completely eliminated, or eliminated over a selected time period. To the extent that 100% inhibition is equivalent to prevention, the term “inhibiting” also includes prevention.

The term “subject” includes living organisms such as humans, monkeys, cows, sheep, horses, pigs, cattle, goats, dogs, cats, mice, rats, cultured cells, and transgenic species thereof. In a preferred embodiment, the subject is a human for carrying out the described methods of preventing HO and/or treating a subject with bone trauma.

The terms “locally administering” or “local administration” refer to the administration of an agent at or substantially near the site at which one wishes to prevent or inhibit HO. Local administration of an agent produces a local, rather than a systemic or global effect, e.g. on proprioception or motor function. As a non-limiting example, intramuscular injection of an agent near the site of bone trauma is local administration.

The term “injury” includes physical trauma, as well as a localized infection or a localized disease process, such as the spontaneous development of a bone spur or heterotopic ossification at a site. The term “injury” includes a surgical procedure, such as implanting or removing an orthopedic device, or a deep bone infection as well. “Inhibiting,” “retarding,” “reducing,” and “impeding” bone growth are intended for use as either equivalent terms or terms designating varying degrees of prevention of inappropriate bone growth. Thus, “inhibiting bone growth” refers to the administration of an agent under conditions, e.g. concentration, rate and/or release of the agent and/or its administration length and/or conditions, such that the amount of inappropriate bone growth is less than the amount that is observed when the agent is not administered (i.e., at least 10% less, and preferably at least 20%, 30%, 40%, 50%, 60%, 70%, 80%, 90%, 95%, or more, including 100% (no inappropriate bone growth)).

As used herein, the terms “pharmaceutically acceptable,” “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The active ingredient can be mixed with excipients which are pharmaceutically acceptable and compatible with the active ingredient and in amounts suitable for use in the therapeutic methods described herein.

Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

The term, “co-administered” means two or more drugs are given to a patient at approximately the same time or in close sequence so that their effects run approximately concurrently or substantially overlap. This term includes sequential as well as simultaneous drug administration.

“Pharmaceutically acceptable carrier” includes any and all solvents, dispersion media, coatings, antibacterial and antifungal agents, isotonic and absorption delaying agents, and the like which are compatible with the activity of the compound and are physiologically acceptable to the subject.

“Administering” includes routes of administration which allow the compositions of the invention to perform their intended function, e.g., preventing HO. Specifically encompassed within the term are injection of an agent preparation and implantation of an agent delivery composition or device (e.g., an osmotic pump or other delivery device).

“Effective amount” includes those amounts of proprioception inhibitor or botulinum toxin which inhibit or prevent inappropriate bone growth or HO as described herein.

The administration of an agent “at a site of injury” means locally administering the agent so that it may be in direct contact with injured bone or muscle in contact with injured bone or muscle in contact with a site at which inappropriate bone growth is desired to be inhibited or prevented. Where bone injury or trauma is involved, the agent can be locally administered at a location proximal to the injured bone, so that the agent can produce the desired or stated therapeutic effect, e.g. reduce bone growth (including inappropriate and heterotopic growth) at the site.

An agent “formulated for controlled release” means that it may be formulated so that it will be released over an extended period of time relative to release of the agent not in such a formulation when administered according to the methods described herein.

An agent is said to be “appended” to a polymer when the agent may be bonded to the polymer as a side chain or side group, but is not part of the polymer backbone.

An agent is said to be “entrapped or dispersed in a polymeric matrix” when it is located within the matrix of a polymer such that it can be released in a controlled fashion when placed within the body.

The “release” of an agent refers to the delivery of an agent in a form that may be bioavailable and/or free, and includes the degradation of a polymer where the agent may be incorporated into, or appended to, the backbone.

The term “release” also includes the degradation of a polymer that entraps agent molecules within its matrix, thereby allowing the free agent to make direct contact with the surrounding tissue or bone. The term “release” also encompasses administration of an agent in a form that may be immediately bioavailable, i.e. not a sustained release formulation.

A substance is said to be “resorbable”, e.g. “bioresorbable”, when its material is capable of being absorbed by, and integrated into, a system, e.g. the living system, when placed into it or when created and subsequently placed in the system.

As used herein, the term “dispersed through the polymer matrix” means that an agent or compound may be located within a matrix, for example a polymer by mixing, spreading, sprinkling, thoroughly mixing, physically admixing, or dispersing in the polymer matrix, among others, so that it may be released in a controlled manner over a period of time when placed in a system, e.g. within a living host.

The term “transient paralysis” refers to the reversible inhibition of motor function induced by local administration of a pharmacological agent.

Proprioception Inhibitor

In various embodiments, the methods described herein involve the administration of one or more proprioception inhibitors to inhibit or prevent HO. Inhibitors of proprioception activities are known in the art. Proprioception inhibitors include, but are not limited to, inhibitors of small-diameter sensory fibers including lidocaine, bupivicaine, and veratridine, saxitoxin, Clostridium botulinum toxin, type A, and other botulinum toxin preparations. Inhibitors can inhibit, for example, the proprioceptive neuronal pathways that would otherwise lead to activation of mesenchymal progenitors in the adjacent soft tissues to form heterotopic bone. Botulinum toxin, e.g., botulinum toxin A is, in addition to its well known neurotoxic effects, a proprioception inhibitor (see, e.g., Manni et al., “Effect of Botulinum Toxin on Extraocular Muscle Proprioception,” Doc Ophthalmol 72: 189-198 (1989), which is hereby incorporated by reference).

Thalhammer et al. (supra) describes quantitative behavioral testing that established a reproducible measure of differential functional blockade during regional anesthesia. Methods for assessment of the neurologic status in veterinary neurology were adapted for the rat and used to monitor functional changes separately during a sciatic nerve block. Sprague-Dawley rats were acclimated to laboratory routine before the study so that lidocaine (0.1 ml, 1%) could be injected near the sciatic notch without any chemical restraint. Proprioceptive integrity was evaluated by assessing postural reactions including the “hopping” response and the “tactile placing” response. A scale of functional deficit grades the inhibition of proprioception as 0 (normal), 1 (slightly impaired), 2 (severely impaired), or 3 (absent).

Hopping response: For this measure of proprioception, a rat is placed with the hind legs on a supporting surface and the front half of the animal is lifted off the ground (held upright by the evaluator). One hind leg at a time is lifted off the ground, and the animal's body is moved laterally. As soon as this happens, the animal normally hops with the weight-bearing limb in the direction of movement to avoid falling over. Delays in the response, graded on the 0-3 scale, are indicative of proprioceptive inhibition. Thus, with a primarily proprioceptive impairment, the hopping response is delayed, and the magnitude of passive lateral movement must be greater to elicit a response. With a primarily motor impairment, there is a prompt response after initiation of a lateral movement, but the response is weaker than normal and the follow-through of the movement is impaired. Thus, the hopping assay can also help in distinguishing proprioceptive from motor inhibition.

Tactile placing response: For this measure of proprioception, a rat is kept in a normal resting posture, and the toes of one foot are flexed with their dorsi placed onto the supporting surface. The ability of the animal to reposition the toes is evaluated on the same 0-3 scale as the hopping assay. is the ability to reposition the knuckled toes such that the plantar surface of the foot rests flat on the support surface.

In the assays described by Thalhammer et al., proprioceptive impairment was detected with lidocaine injection near the sciatic nerve. Complete absence of proprioception occurred from 10 to 30 min (n=9) as measured by both the hopping and tactile placing assays. Function was fully recovered by 120 min. Thalhammer et al. also describes further methods for the evaluation of motor function impairment. The Thalhammer et al. approach to the measurement of proprioceptive versus motor impairment was applied by Vladimirov et al., “Neurophysiologic Actions and Neurological Consequences of Veratridine on the Rat Sciatic Nerve,” Anesthesiology 86(4):945-956 (1997), which is hereby incorporated by reference in its entirety) to establish the anti-proprioceptive effects of veratridine.

At a minimum, a proprioception inhibitor preparation will inhibit HO, e.g., in the transcortical defect murine model for bone growth described herein. This assay includes generating a transcortical defect (TCD), which models the osteogenic response to skeletal trauma. (See the Examples herein). A proprioceptor inhibitor as the term is used herein will inhibit HO in this assay at least 25% as much as the maximally effective dose of Botulinum toxin serotype A. When tested in this assay, C. botulinum toxin, type A injection of the calf muscle group comprising the gastrocnemius, plantaris, and soleus muscles profoundly inhibits osteogenic bone formation distal and proximal to the TCD. In contrast, the control saline-injected mice shows profound osteogenesis. This assay allows for measurement of the effects of agents in the inhibition of inappropriate bone growth and/or HO.

A proprioception inhibitor can be used for the inhibition of heterotopic ossification (HO) in a subject in need thereof, for the treatment of bone trauma, and for the inhibition or prevention of HO. A proprioception inhibitor can also be used for the preparation of a medicament for the prevention of HO and for the treatment of bone trauma. The proprioception inhibitor can be selected from the group consisting of inhibitors of small-diameter sensory fibers, saxitoxin, Clostridium botulinum toxin, type A, and other botulinum toxin preparations. The proprioception inhibitor can be Clostridium botulinum toxin, type A. The inhibitor of small-diameter sensory fibers can be selected from the group consisting of lidocaine, bupivicaine, and veratridine.
Botulinum toxin for prevention or inhibition of inappropriate bone growth:

As reported in U.S. Patent Application Publication 2006/0024794 A1 to Williams et al., which is hereby incorporated by reference in its entirety, botulinum toxins have been used in clinical settings for the treatment of neuromuscular disorders characterized by hyperactive skeletal muscles. In 1989 a botulinum toxin serotype A complex was approved by the U.S. Food and Drug Administration (FDA) for the treatment of blepharospasm, strabismus and hemifacial spasm. Subsequently, a botulinum toxin serotype A was also approved by the FDA for the treatment of cervical dystonia and for the treatment of glabellar lines. Clinical effects of peripheral intramuscular botulinum toxin serotype A are usually seen within one week of injection. The success of botulinum toxin serotype A to treat a variety of clinical conditions has led to interest in other botulinum toxin serotypes. A botulinum toxin serotype B was approved for the treatment of cervical dystonia.

Two commercially available botulinum serotype A preparations for use in humans are BOTOX™ available from Allergan, Inc., of Irvine, Calif., and Dysport® available from Beaufour Ipsen, Porton Down, England. A Botulinum toxin serotype B preparation (MyoBloc®) is available from Elan Pharmaceuticals of San Francisco, Calif.

Intramuscular botulinum toxin has been used in the treatment of tremor in patients with Parkinson's disease. See Marjama-Jyons et al., “Tremor-Predominant Parkinson's Disease,” Drugs & Aging 16(4); 273-278:(2000), which is hereby incorporated by reference in its entirety.

The typical duration of symptomatic relief from a single intramuscular injection of botulinum toxin serotype A averages about three months, although significantly longer periods of therapeutic activity have been reported. It is known that botulinum toxin serotype A can have an efficacy for up to 12 months (Naumann et al., “Botulinum Toxin Type A in the Treatment of Focal, Axillary and Palmar Hyperhidrosis and Other Hyperhidrotic Conditions,” European J Neurology 6 (Supp 4): S111-S1150 (1999), which is hereby incorporated by reference in its entirety), and in some circumstances for as long as 27 months (Ragona et al., “Management of Parotid Sialocele with Botulinum Toxin”, The Laryngoscope 109:1344-1346 (1999), which is hereby incorporated by reference in its entirety). However, the usual duration of an intramuscular injection of BOTOX™ (Clostridium botulinum toxin, type A) is typically about 3 to 4 months.

Seven generally immunologically distinct botulinum neurotoxins have been characterized: botulinum neurotoxin serotypes (types) A, B, C1, D, E, F and G. These serotypes are distinguished by neutralization with serotype-specific antibodies. The different serotypes of botulinum toxin vary in the animal species that they affect and in the severity and duration of the paralysis they evoke. For example, it has been determined that botulinum toxin serotype A is 500 times more potent, as measured by the rate of paralysis produced in the rat, than is botulinum toxin serotype B. Additionally, botulinum toxin serotype B has been determined to be non-toxic in primates at a dose of 480 U/kg which is about 12 times the primate LD50 for botulinum toxin serotype A (Moyer et al., “Botulinum Toxin Serotype B: Experimental and Clinical Experience,” Ch. 6, pp. 71-85 of “Therapy With Botulinum Toxin,” edited by Jankovic et al. Marcel Dekker, Inc. (1994), which is hereby incorporated by reference in its entirety). Botulinum toxin apparently binds with high affinity to cholinergic motor neurons, is translocated into the neuron and blocks the release of acetylcholine.

The molecular weight of the botulinum toxin protein molecule, for all seven of the known botulinum toxin serotypes, is about 150 kD. Interestingly, the botulinum toxins are released by Clostridial bacterium as complexes comprising the 150 kD botulinum toxin protein molecule along with associated non-toxin proteins. Thus, the botulinum toxin serotype A complex can be produced by Clostridial bacterium as 900 kD, 500 kD and 300 kD forms. Botulinum toxin serotypes B and C1 are apparently produced as only a 700 kD or 500 kD complex. Botulinum toxin serotype D is produced as both 300 kD and 500 kD complexes. Finally, botulinum toxin serotypes E and F are produced as only approximately 300 kD complexes. The complexes (i.e. molecular weight greater than about 150 kD) are believed to contain a non-toxin hemagglutinin protein and a non-toxin and non-toxic nonhemagglutinin protein. These two non-toxin proteins (which along with the botulinum toxin molecule comprise the relevant neurotoxin complex) may act to provide stability against denaturation to the botulinum toxin molecule and protection against digestive acids when toxin is ingested. Additionally, it is possible that the larger (greater than about 150 kD molecular weight) botulinum toxin complexes may result in a slower rate of diffusion of the botulinum toxin away from a site of intramuscular injection of a botulinum toxin complex. Each of the botulinum toxin serotypes is contemplated for use in the methods of inhibiting or preventing inappropriate bone growth as described herein.

An approved, commercially available botulinum toxin-containing pharmaceutical composition sold under the trademark BOTOX™ (available from Allergan, Inc., of Irvine, Calif.) consists of a purified botulinum toxin serotype A complex, albumin and sodium chloride packaged in sterile, vacuum-dried form. The botulinum toxin serotype A is made from a culture of the Hall strain of Clostridium botulinum grown in a medium containing N-Z amine and yeast extract. The botulinum toxin serotype A complex is purified from the culture solution by a series of acid precipitations to a crystalline complex consisting of the active high molecular weight toxin protein and an associated hemagglutinin protein. The crystalline complex is re-dissolved in a solution containing saline and albumin and sterile filtered (0.2 μm) prior to vacuum-drying. The vacuum-dried product is stored in a freezer at or below −5° C. BOTOX™ (Clostridium botulinum toxin, type A) can be reconstituted with sterile, non-preserved saline prior to intramuscular injection or other local administration. Each vial of Botox™ contains about 100 units (U) of Clostridium botulinum toxin serotype A purified neurotoxin complex, 0.5 milligrams of human serum albumin and 0.9 milligrams of sodium chloride in a sterile, vacuum-dried form without a preservative.

To reconstitute vacuum-dried BOTOX™ (Clostridium botulinum toxin, type A), sterile normal saline without a preservative; (0.9% Sodium Chloride Injection) is used by drawing up the proper amount of diluent in the appropriate size syringe. Since BOTOX™ (Clostridium botulinum toxin, type A) may be denatured by bubbling or similar violent agitation, the diluent is gently injected into the vial. For sterility reasons BOTOX™ (Clostridium botulinum toxin, type A) is preferably administered within four hours after the vial is removed from the freezer and reconstituted. During these four hours, reconstituted BOTOX™ (Clostridium botulinum toxin, type A) can be stored in a refrigerator at about 2° C. to about 8° C. Reconstituted, refrigerated BOTOX™ (Clostridium botulinum toxin, type A) has been reported to retain its potency for at least about two weeks (Sloop et al., “Reconstituted Botulinum Toxin Type A Does Not Lose Potency in Humans If It Is Refrozen or Refrigerated for 2 Weeks Before Use,” Neurology 48:249-53 (1997), which is hereby incorporated by reference in its entirety.

Generally, commercial botulinum toxins are produced by establishing and growing cultures of Clostridium botulinum, E. coli cells or recombinantly engineered yeast cells in a fermenter and then harvesting and purifying the fermented mixture in accordance with known procedures. All the botulinum toxin serotypes are initially synthesized as inactive single chain proteins. To be converted into their active forms, the single chain botulinum toxins are subsequently nicked by proteases, e.g. trypsin.

Heterotopic Ossification

As described in U.S. Patent Application Publication No. 20050249697 to Uhrich et al., which is hereby incorporated by reference in its entirety, HO involves unwanted bone growth that may be characterized by inappropriate differentiation of cells into bone-forming cells. This condition leads to bone formation, usually near joints, where the bone formation often limits the mobility of the joint. HO may follow neurological injury and direct injury to soft tissue such as muscles or connective tissue around the joint in which HO later develops. In the case of an elbow fracture or dislocation, the subsequent incidence of HO at the elbow is said to approach 90%. It may be desirable as well to inhibit bone growth following a bone fracture because new bone growth prior to setting may impair proper healing of the fracture site afterwards. Following surgical procedures, for instance following a spinal laminectomy, inappropriate new bone growth can impinge on the spinal cord and cause complications such as pain, numbness and paralysis.

There are three recognized etiologies of HO: traumatic, neurogenic, and genetic. Traumatic HO typically follows fractures, dislocations, operative procedures, and severe burns. Most commonly, HO is seen around the hip after fracture and open reduction-internal fixation (ORIF) procedures or total hip arthroplasties (THA) (Balboni et al., “Heterotopic Ossification: Pathophysiology, Clinical Features, and the Role of Radiotherapy for Prophylaxis,” Int J Radiation Oncology Biol Phys 65(5): 1289-1299 (2006), which is hereby incorporated by reference in its entirety).

HO is a frequent secondary complication following total hip arthroplasty (THA), open reduction internal fixation (ORIF) of acetabular fractures, spinal fusions, amputation, fracture, and soft tissue releases about the hips. As well, HO is often associated with pathologies such as traumatic brain injury (TBI), spinal cord injury (SCl), infections of the central nervous system (CNS), tumors, strokes, tetanus, polio, tabes dorsalis, multiple sclerosis, and selective posterior rhizotomy. The presence of idiopathic muscle spasticity is also associated with the development of HO. While many patient populations are at risk of developing HO, the incidence varies within each population. For example, in traumatic etiologies, HO incidence following THA is approximately 53% (Shehab et al., “Heterotopic Ossification,” J Nucl Med 43(3):346-53 (2002), which is hereby incorporated by reference in its entirety); in ORIF of acetabular fractures HO incidence is estimated to be about 25% (Giannoudis et al., “Operative Treatment of Displaced Fractures of the Acetabulum. A Meta-analysis,” J Bone Joint Surg Br 87(1):2-9 (2005), which is hereby incorporated by reference in its entirety); following amputation from traumatic injury, such as those endured by military personnel, HO incidence is 63% (Potter et al., “Heterotopic Ossification Following Traumatic and Combat-related Amputations. Prevalence, Risk Factors, and preliminary Results of Excision,” J Bone Joint Surg Am 89(3):476-86 (2007), which is hereby incorporated by reference in its entirety); and in severe burns, HO incidence is from 1-3%. HO from neurogenic causes such as SCI occurs in 20 to 30% of the SCI population (Shehab et al., “Heterotopic Ossification,” J Nucl Med 43(3):346-53 (2002), which is hereby incorporated by reference in its entirety), while HO following traumatic brain injury occurs from 10-20% of patients (Garland D E., “A Clinical Perspective on Common Forms of Acquired Heterotopic Ossification,” Clin Orthop Relat Res (263):13-29 (1991), which is hereby incorporated by reference in its entirety). Clinically significant HO is reported in 10-20% of HO cases (Garland D E., “A Clinical Perspective on Common Forms of Acquired Heterotopic Ossification,” Clin Orthop Relat Res (263):13-29 (1991), which is hereby incorporated by reference in its entirety). As would be expected, ossification of soft tissue is likely to cause severe restrictions in joint mobility, precipitate entrapment of peripheral nerves, and induce formation of pressure ulcers all of which contribute to severe pain and debilitation (Shehab et al., “Heterotopic Ossification,” J Nucl Med 43(3):346-53 (2002), which is hereby incorporated by reference in its entirety).

Epidemiologic assessment of the incidence of HO has been focused on its association with individual pathologies, and the incidence of HO varies within each population. Clinically significant HO, in which joint motion is restricted physically or by extreme pain, has been reported in 10-20% of HO cases (Garland D E., “A Clinical Perspective on Common Forms of Acquired Heterotopic Ossification,” Clin Orthop Relat Res (263):13-29 (1991), which is hereby incorporated by reference in its entirety). Table 1 lists the main populations that are impacted by HO, demonstrating a total of more than 1.6 million HO cases each year in the United States. Applying the 10 to 20% estimate, we conservatively estimate that 160,000 to 333,000 patients per year, in the U.S. alone, develop HO severe enough to require surgical intervention.

TABLE 1
Populations impacted by heterotopic ossification
(HO) in the United States

| | Total | HO | HO |
|---|---|---|---|
| Etiology | Patients US | Incidence | Patients |
| Total Hip Arthroplasty | 193,000 ^{a} | 53% | 106,000 |
| Traumatic Brain Injury | 1.4 million ^{b} | 10-20% | 210,000 |
| Spinal Cord Injury | 11,000 ^{b} | 20-30% | 2,750 |
| Amputation | 2.1 million ^{c} | 63% | 1,323,000 |
| Burns | 1.1 million ^{b} | 1-3% | 16,500 |
| ORIF Acetabular Fracture | 11,250 ^{a} | 90% | 10,125 |
| | | Total Per Year: | 1,668,400 |

^{a} American Academy of Orthopaedic Surgeons,
^{b} Center for Disease Control,
^{c} NHIS95

Prophylactic treatment of HO is currently optional, and involves several different approaches of varying side effect risk and cost. One low cost treatment modality is the use of nonsteroidal anti-inflammatory drugs (NSAIDs) (Burd et al., “Indomethacin Compared with Localized Irradiation for the Prevention of heterotopic Ossification Following Surgical Treatment of Acetabular Fractures,” J Bone Joint Surg Am 83-A(12):1783-8 (2001), which is hereby incorporated by reference in its entirety). NSAID therapy typically consists of a 6 week course of medication with multiple daily doses (Garland D E., “A Clinical Perspective on Common Forms of Acquired Heterotopic Ossification,” Clin Orthop Relat Res (263):13-29 (1991), Burd et al., “Indomethacin Compared with Localized Irradiation for the Prevention of heterotopic Ossification Following Surgical Treatment of Acetabular Fractures,” J Bone Joint Surg Am 83-A(12):1783-8 (2001), which are hereby incorporated by reference in their entirety). Prolonged NSAID use can lead to gastrointestinal bleeding, which can be a serious side effect in patients who are also, for example, on anti-coagulation therapy for deep vein thrombosis or cardiac arythmia (Balboni et al., “Heterotopic Ossification: Pathophysiology, Clinical Features, and the Role of Radiotherapy for Prophylaxis,” Int J Radiat Oncol Biol Phys 65(5):1289-99 (2006), Chao et al., “Treatment of Heterotopic Ossification,” Orthopedics 30(6):457-64;quiz 465-6 (2007), which are hereby incorporated by reference in their entirety). Additionally, extended NSAID exposure has been found to reduce bone ingrowth on cementless stems in THA, and to increase the rate of nonunions following fracture (Balboni et al., “Heterotopic Ossification: Pathophysiology, Clinical Features, and the Role of Radiotherapy for Prophylaxis,” Int J Radiat Oncol Biol Phys 65(5):1289-99 (2006), Chao et al., “Treatment of Heterotopic Ossification,” Orthopedics 30(6):457-64;quiz 465-6 (2007), which are hereby incorporated by reference in their entirety), which is particularly relevant in the context of trochanteric nonunions following THA.

Anti-resorptive bisphosphonates have also been used to treat HO, yet a six month course of medication was found to merely prevent mineralization of the matrix. In at least one report, upon completion of bisphosphonate intervention, the matrix underwent uninhibited mineralization and recurrence of an HO nodule (Vanden Bossche et al., “Heterotopic Ossification: A Review,” J Rehabil Med 37(3):129-36 (2005), Balboni et al., “Heterotopic Ossification: Pathophysiology, Clinical Features, and the Role of Radiotherapy for Prophylaxis,” Int J Radiat Oncol Biol Phys 65(5):1289-99 (2006), which are hereby incorporated by reference in their entirety).

In contrast to systemic NSAIDs or bisphosphonate therapies, the prophylactic use of radiation therapy represents a non-invasive HO therapy in the limited subset population of THA patients. Radiation therapy in this population is generally successful mainly due to the site-specific nature of HO nodule formation in the abductor compartment following this specific surgery (Garland D E., “A Clinical Perspective on Common Forms of Acquired Heterotopic Ossification,” Clin Orthop Relat Res (263):13-29 (1991), which is hereby incorporated by reference in its entirety). However, because the location of HO can be difficult to predict for non-THA etiologies, the application of radiation treatment in idiopathic HO has not been widely investigated. The predominant concern in using radiotherapy as a treatment modality for HO is the potential for carcinogenic sequelae. Other side effects of radiotherapy include trochanteric nonunion (Kienapfel et al., “Prevention of Heterotopic Bone Formation After Total Hip Arthroplasty: A Prospective Randomised Study Comparing Postoperative Radiation Therapy With Indomethacin Medication,” Arch Orthop trauma Surg 119(5-6):296-302 (1999), which is hereby incorporated by reference in its entirety), or potential damage to reproductive organs. In addition, the high cost of this radiotherapy may be prohibitive for routine prophylactic application (Oertel et al., “Prophylaxis of Heterotopic Ossification in Patients Sedated After Polytrauma: Medical and Ethical Considerations,” Strahlenther Onkol 184(4):212-7 (2008), which is hereby incorporated by reference in its entirety). The comparative efficacy of NSAIDs and radiation therapy for HO following THA has been extensively studied. Among published studies, variability exists regarding dose, timing of treatment (pre- or post-operatively), duration of treatment and type of medication. In a meta-analysis of eight studies, radiation provided a decreased risk of clinically significant HO compared to NSAIDs, yet the absolute difference was small (1.2%). As well, a dose response relationship showed that only radiation doses higher than 6 Gy were more effective than NSAIDs treatment (Chao et al., “Treatment of Heterotopic Ossification,” Orthopedics 30(6):457-64;quiz 465-6 (2007), which is hereby incorporated by reference in its entirety).

Surgery is currently the only definitive treatment to remove mineralized HO. The timing of the surgical removal is dependent on the etiology of HO. For traumatic HO, the nodule should not be removed prior to six months after onset. In SCI patients, surgery to remove HO should be delayed from 1.5 to two years, while in TBI8 patients, restoration of motor recovery should be the guide for HO removal, typically 1.5 years after the injury (Garland D E., “A Clinical Perspective on Common Forms of Acquired Heterotopic Ossification,” Clin Orthop Relat Res (263):13-29 (1991), which is hereby incorporated by reference in its entirety). Postoperative complications following HO removal tend to occur with more frequency compared to routine orthopaedic procedures (Garland D E., “A Clinical Perspective on Common Forms of Acquired Heterotopic Ossification,” Clin Orthop Relat Res (263):13-29 (1991), which is hereby incorporated by reference in its entirety). The surgical removal of HO is expensive, requires general anesthesia and includes potential complications such as deep vein thrombosis, infection and hemorrhage (Vanden Bossche et al., “Heterotopic Ossification: A Review,” J Rehabil Med 37(3):129-36 (2005), which is hereby incorporated by reference in its entirety). In addition, recurrence of HO after surgical removal of the bone mass occurs in a high percentage of patients. Therefore, careful resection of the HO nodule is imperative in order to reduce the likelihood of HO recurrence after surgical removal.

In the pathophysiology of HO, osteoid formation commonly follows an inflammatory phase characterized by local swelling, pain, erythema and sometimes fever and joint restriction (Strakowski et al., “Upper Limb Musculoskeletal Pain Syndromes,” In: Buschbaker et al. editor(s). Physical Medicine and Rehabilitation. 2nd Edition. Philadelphia: WB Saunders Company, 779 (1996), Yarkony et al., “Rehabilitation of Patients with Spinal Cord Injuries,” In: Buschbaker et al. editor(s). Physical Medicine and Rehabilitation. 2nd Edition. Philadelphia: WB Saunders Company, 1162-63 (1996)). Two to six weeks prior to its detectability using plain radiography, bone scintigraphy and ultrasonography can reliably diagnose HO (Orzel et al., “Heterotopic Bone Formation: Clinical, Laboratory, and Imaging Correlation,” J Nuclear Medicine 26(2):125-32 (1985), Freed et al., “The Use of the Three-phase Bone Scan in the Early Diagnosis of Heterotopic Ossification (HO) and in the Evaluation of Didronel Therapy,” Paraplegia 20(4):208-16 (1982), Pistarini et al., “The Echographic Diagnosis of Neurogenic Paraostearthropathies in Myelosis Patients,” Giornale Italiano di Medicina del Lavoro 15(5-6):159-63 (1993), which are hereby incorporated by reference in their entirety), providing the earliest opportunity for acute treatment aimed at minimizing the eventual radiographic grade. Bony maturation usually occurs by six months. Thereafter, regression of HO rarely occurs (Garland D E., “A Clinical Perspective on Common Forms of Acquired Heterotopic Ossification,” Clin Orthop Relat Res (263):13-29 (1991), which is hereby incorporated by reference in its entirety).

Overall, around 20% of HO results in functional impairment (Buschbacher R., “Heterotopic Ossification: A Review. Critical Reviews in Physical Medicine and Rehabilitation,” 4:199-213 (1992), which is hereby incorporated by reference in its entirety) with many patients progressing to severe disability (Subbarao et al., “Heterotopic Ossification: Diagnosis and Management, Current Concepts and Controversies,” J Spinal Cord Medicine 22(4):273-83 (1999), which is hereby incorporated by reference in its entirety). In addition to NSAIDS, other potentially beneficial agents include coumadin/warfarin (Buschbacher et al., “Warfarin in Prevention of Heterotopic Ossification,” American J Physical Medicine Rehabilitation 71(2):86-91 (1992), which is hereby incorporated by reference in its entirety) and salicylates (Mital M A et al., “Ectopic Bone Formation in Children and Adolescents with Head Injuries: Its Management,” J Pediatric Orthopedics 7(1):83-90 (1987), which is hereby incorporated by reference in its entirety). Pharmacological treatment of early HO is most likely to be effective if commenced prior to its detectability on plain radiography (Orzel et al., “Heterotopic Bone Formation: Clinical, Laboratory, and Imaging Correlation,” J Nuclear Medicine 26(2):125-32 (1985), Banovac K., “The Effect of Etidronate on Late Development of Heterotopic Ossification After Spinal Cord Injury,” J Spinal Cord Medicine 23(1):40-4 (2000), which are hereby incorporated by reference in their entirety).

Dosage and Administration

Administration of the compositions of the present invention to a subject to be treated can be carried out using known procedures, at dosages and for periods of time effective to treat the condition in the subject.

A variety of routes of administration are possible including, but not necessarily limited to intramuscularly (parenteral), by implantation, or intralesionally. They may be administered alone or with suitable pharmaceutical carriers, and can be in solid or liquid form such as, tablets, capsules, powders, solutions, suspensions, or emulsions. An agent, e.g., a proprioception inhibitor or botulinum toxin preparation can be administered alone, to prevent HO. However, combination therapy may also be used.

An appropriate composition comprising the compound to be administered can be prepared in a physiologically acceptable vehicle or carrier and optional adjuvant and preservatives. For solutions or emulsions, suitable carriers include, for example, aqueous or alcoholic/aqueous solutions, emulsions or suspensions, including saline and buffered media, sterile water, creams, ointments, lotions, oils, pastes and solid carriers. Parenteral vehicles or carriers can include, for example, sodium chloride solution, Ringer's dextrose, dextrose and sodium chloride, lactated Ringer's or fixed oils. (See generally, Remington's Pharmaceutical Science, 16th Edition, Mack, Ed. (1980), which is hereby incorporated by reference in its entirety).

An example of a pharmaceutically acceptable carrier is buffered normal saline (0.15M NaCl). The use of such media and agents for pharmaceutically active substances is well known in the art. Except insofar as any conventional medium or agent is incompatible with the therapeutic compound, use thereof in the compositions suitable for pharmaceutical administration is contemplated. Supplementary active compounds can also be incorporated into the compositions.

Additionally, another special aspect of the present invention is to provide a pharmaceutical composition that would be useful in the treatment and prevention of heterotopic ossification and other diseases involving undesired bone formation. Such a composition would significantly add to the options that now are available in the treatment of HO, and would lack the side effects of the NSAIDs and the radiotherapy, which at present are the alternative methods of treatment.

A method of administering a proprioception inhibitor to a subject under conditions effective to inhibit or prevent HO is described. Administration of the proprioception inhibitor can lower, reduce or inhibit HO levels or incidence, for example, in a subject having had hip replacement. By “lower,” “reduced” or “inhibited” in the context of inhibiting or preventing HO is meant a statistically significant decrease in such level or incidence relative to the absence of proprioception inhibitor administration. The decrease can be, for example, at least 10%, at least 20%, at least 30%, at least 40% or more, and is preferably down to a level accepted as within the range of normal for a subject without HO.

Efficacy of treatment or prevention of disease can be assessed, for example by measuring disease progression, disease remission, symptom severity, reduction in pain, quality of life, dose of a medication required to sustain a treatment effect, level of a disease marker or any other measurable parameter appropriate for a given disease being treated or targeted for prevention. It is well within the ability of one skilled in the art to monitor efficacy of treatment or prevention by measuring any one of such parameters, or any combination of parameters. A treatment or preventive effect is evident when there is a statistically significant improvement in one or more parameters of disease status, or by a failure to worsen or to develop symptoms where they would otherwise be anticipated. As an example, a favorable change of at least 10% in a measurable parameter of disease, and preferably at least 20%, 30%, 40%, 50% or more can be indicative of effective treatment. Efficacy for a given proprioception inhibitor, botulinum toxin or formulation of such inhibitor or toxin can also be judged using an experimental animal model for the given disease as known in the art, e.g., the murine transcortical defect model described herein. When using an experimental animal model, efficacy of treatment is evidenced when a statistically significant reduction in a marker or symptom is observed.

As noted above, combination of a proprioception inhibitor with another therapeutic agent is specifically provided for herein. In a combination therapy, the anti-proprioception agent is administered before, during, or after commencing therapy with another agent, as well as any combination thereof, i.e., before and during, before and after, during and after, or before, during and after commencing the combination therapy. Combination agents can include, for example, other agents used for the inhibition of HO such as radiation therapy or NSAIDS.

An effective amount of the therapeutic compound necessary to achieve a therapeutic effect may vary according to factors such as the age, sex, and weight of the subject, and the ability of the therapeutic compound to treat the foreign agents in the subject. Dosage regimens can be adjusted to provide the optimum therapeutic response. For example, several divided doses may be administered or the dose may be proportionally reduced as indicated by the exigencies of the therapeutic situation. For example, in a mouse model, dosages of the active substance (e.g., Clostridium botulinum toxin, type A) may be 2.0 units/100 g body weight in an injection volume of 200 to prevent or inhibit HO.

Dosage is calculated as 2 units/100 g bodyweight. However, studies in rat were scaled by muscle mass. Therefore, with the above dose and a 22 g mouse, 0.44 units is used for a 22 g mouse. The average calf muscle groups wet weight is about 120 mg, which works out to 3.67 units/gram muscle mass. Doses of up to 60 units per 100 g bodyweight have been used in children (see Sarioglu et al., “The Use of Botulinum Toxin Type A Treatment in Children With Spasticity,” Pediatr Neurol 29(4):299-301 (2003), which is hereby incorporated by reference in its entirety). Therefore, it is estimated that 2 U/100 g bodyweight provides a baseline to establish effective treatment dosing in humans. However, it is anticipated that any dosing that has previously been associated with reduced muscle function and/or decreased proprioception and/or decreased spasticity would be effective.

Administration is local, at the site where one expects or wishes to prevent inappropriate bone growth, e.g., HO. The frequency and individual amount of dosages for either therapeutic or maintenance/prophylactic uses will also depend, for example, on the in vivo half-life of the proprioception inhibitor used. Thus, more frequent dosing is appropriate where the half-life is shorter, and vice versa. One of skill in the art can measure the in vivo half-life for a given proprioception inhibitor.

Formulations

Provided herein are formulations for the delivery of a proprioception inhibitor, including, but not limited to a botulinum toxin where HO is inhibited or prevented. Injected or locally administered C. botulinum toxin type A has a long resident half-life. However, it is specifically contemplated that a formulation includes an extended release form of this or another effective agent.

Methods and compositions described herein include proprioception inhibitor formulations having one or more excipients that enhance the bioavailability of the proprioception inhibitor when administered. Among the excipients are, for example, stabilizing agents, osmotic agents, agents that assist in solubilizing or maintaining the solubility of the drug agents, and viscosity modifiers. Specific excipients and effective combinations, amounts and formulations thereof are described in the following.

In one aspect, provided herein is a formulation of proprioception inhibitor for administration to inhibit or prevent HO, the formulation comprising Clostridium botulinum toxin, type A, in an amount of 2.0 units/100 g body weight in an injection volume of 20 μl in a mouse model. This provides a guide for effective dosing in, e.g., humans; however, the local administration and effect of the agent means that the dosage need not necessarily be keyed to the weight of the individual or subject being treated. In one embodiment, the formulation is administered intramuscularly.

In another embodiment, the formulation is administered by implantation, e.g., implantation of an extended release formulation, e.g., in a polymer or, for example, implantation of a pump, e.g., an osmotic pump. In another embodiment, the formulation is administered intralesionally.

The agent (e.g., proprioception inhibitor) can conveniently be formulated, for example, so that it will be released over a period of at least about 2, about 5, about 12 about 24, or about 48 hours, or over at least about 2, about 5, about 10, about 20, or about 40 days. Preferably, the agent can be formulated so that it can be released over at least about 5 or about 10 days. The agent can also preferably be formulated so that it can be released over a period of about 30 to about 90 days. The agent can be bonded preferably to a polymer through a linkage that is suitable to release the agent when the polymer is administered or implanted. The agent can conveniently be linked to a polymer through a hydrolyzable linkage such as an anhydride or ester linkage. The polymer matrix can comprise a bio-degradable polymer.

In yet another embodiment the proprioception inhibitor can be formulated for controlled release for administration or application at a pre-selected site, such as a site of injury. The proprioception inhibitor, for example, can be entrapped in a matrix formed by a biodegradable monomer(s), oligomer(s), polymer(s), or salt(s) or mixture(s) or blend(s) thereof, appended to or incorporated into a biodegradable polymer backbone of any chemical composition. In a further embodiment the monomer(s), oligomer(s), polymer(s), blend(s), mixture(s) or salt(s) thereof can be incorporated into a film, paste, gel, fiber, chip, powder, tablets, capsules, microparticles, or nanoparticles, among many others. In a specific embodiment they can be incorporated into a microparticle(s). Other extended-release formulations, such as formulations employing liposomes and/or cyclodextrins are specifically contemplated for use in the methods and compositions described herein. The concentration of the proprioception inhibitor present or loaded into an implant, device or dressing will depend on the application and on the period of time required for release.

Additionally, controlled release toxin implants are known (see e.g. U.S. Pat. Nos. 6,306,423 to Donovan et al. and 6,312,708 to Donovan). All of the above patent references are hereby incorporated by reference in their entirety.

### EXAMPLES

The following examples are provided to illustrate embodiments of the present invention but are by no means intended to limit its scope.

### Example 1

### Transient Paralysis of Muscle Using Clostridium botulinum Toxin, Type A, Inhibits Trauma-Induced Stimulation of Intramembranous Bone Formation

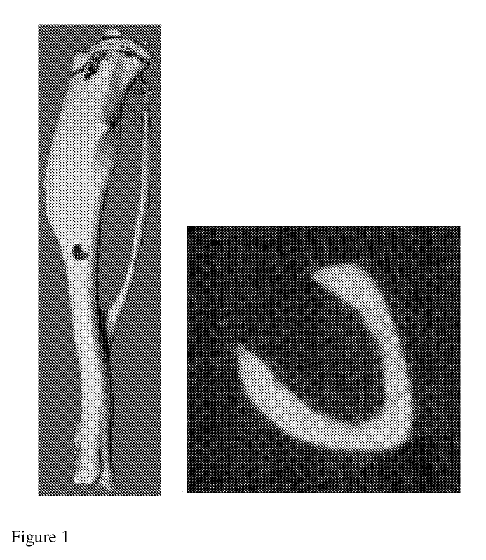

Female C57B1/6 mice (16 wk) were randomly assigned to one of 2 groups (n=4 per group): 1) TCD+saline; 2) TCD+Botox-Calf. On day 0 of the experiment, each mouse was anesthetized with a ketamine/xylazine anesthetic and an incision was be made over the anteromedial surface of the right tibial diaphysis. The muscle was blunt dissected to expose the periosteal surface and a 0.6 mm diameter penetrating hole was created in the medial cortex approximately 1 mm distal from the termination of the tibial tuberosity (FIG. 1). While still anesthetized, all of the mice in group 1 received an injection of 20 μl sterile saline into the gastrocnemius muscle and all of the group 2 mice received a gastrocnemius injection of Clostridium botulinum toxin, type A (“BT×A”) at a dose of 2.0 units/100 g body weight in an injection volume of 20 μl. Following surgery and saline or BT×A injections, all animals underwent high resolution micro-CT scan (Scanco vivaCT 40; 11 μm voxel resolution) to confirm the location of the TCD. A second and third micro-CT scan was performed in all animals at 12 and 21 days, respectively.

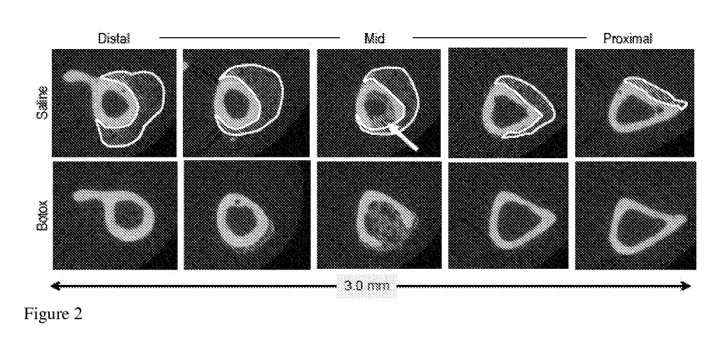

At 12 days post injury, micro-CT scanning of the saline-injected mice showed profound osteogenesis on the periosteal bone surface proximal and distal to the bone trauma (FIG. 2). The micro-CT images of these saline-injected mice were consistent with the interpretation that the osteogenic response is a result of the stimulation of intramembranous bone formation along the length of the diaphyseal bone.

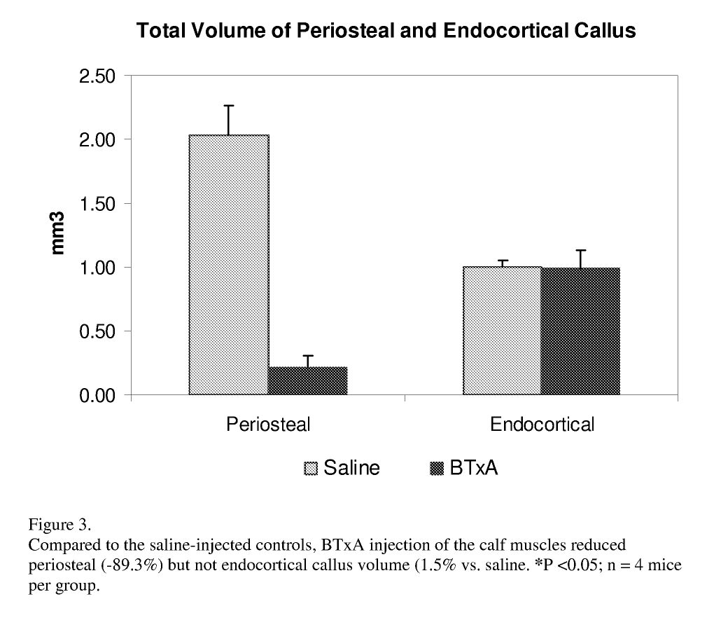

Compared to the intramembranous bone response observed in saline treated mice, BT×A injection of the gastrocnemius muscle profoundly inhibited osteogenic bone formation distal and proximal to the TCD (FIG. 2). Significantly, the only osteogenic tissues observed in BT×A treated animals were immediately adjacent to the TCD or within the defect itself, limiting the osteogenic response to the skeletal trauma by nearly 89.3% (FIG. 3).

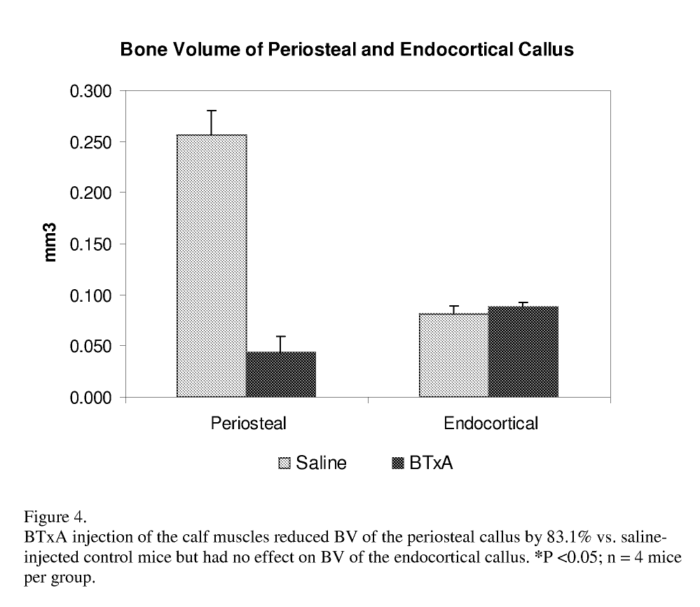

Furthermore, while BT×A injection of the calf muscle profoundly inhibited periosteal osteogenesis, it had no effect on bone formation on endocortical surfaces (percent difference saline vs. BT×A of 1.5%), nor did it affect bone healing or mineralization at the defect site. Similarly, the volume of osteogenic tissue in the periosteal response was also greatly diminished by BT×A injection of the calf muscles as bone volume (BV) in mice treated with BT×A was decreased 83.1% vs. saline-treated animals (FIG. 4; p<0.05). The osteogenic response observed in the BT×A treated animals was limited to the periosteum immediately adjacent to the TCD, within the defect itself, or at the endocortical surface. In summary, BT×A-induced transient muscle paralysis of the calf muscles mitigated the periosteal osteogenic response to the surgically-induced skeletal trauma.

### Example 2

### BT×A does not Directly Inhibit Osteoblast Function

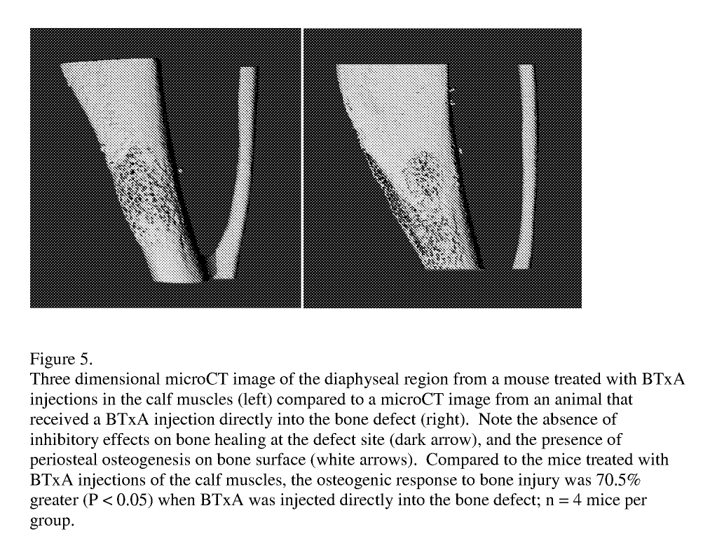

In order to confirm that the inhibitory effects on periosteal osteogenesis were not a consequence of direct inhibitory actions of BT×A on osteoblast function, a control experiment was conducted in which BT×A was injected directly into the bone defect. Compared to mice injected with BT×A into the calf muscles (n=4), direct injection of BT×A into the bone defect had no effect on the osteogenic response to the skeletal trauma (n=4). Indeed, microCT images at 12 days post injury clearly demonstrated normal healing of the cortical bone defect (FIG. 5) and minimal effects on periosteal osteogenesis, which was 70.5% greater (P<0.05) than the osteogenic response in mice with BT×A injected into the calf muscles.

Based on these data, one can conclude that the ability of BT×A to inhibit the periosteal osteogenic response to surgically-induced bone trauma is derived from BT×A-induced inhibition of neuromuscular signaling rather than a direct effect of BT×A on the cellular cascade precipitated by surgical trauma to the cortical bone.

Results from this experiment indicate that transient paralysis of muscle (including inhibition of proprioception and motor function), for example, using Clostridium botulinum toxin, type A is responsible for blocking osteogenic activity during trauma-induced intramembranous bone formation. Additional data (see Example 3) indicate that the inhibition of neuromuscular activity proximal to the bone lesion also blocks trauma-induced osteogenesis. Specifically, transient paralysis of the quadriceps muscle (which inhibits neuronal transmission in transit from the calf) inhibits the trauma-induced osteogenic response. Without wishing to be bound by theory, and based on these data, the inhibitory effects of botulinum toxin, type A can result from the drug's ability to block proprioceptive neuronal pathways that would otherwise activate mesenchymal progenitors in the adjacent soft tissues to form heterotopic bone. Thus, other agents that can block or inhibit proprioception can also be effective inhibitors of heterotopic bone formation.

The experiments detailed above describe the superimposed transient paralysis of the calf muscle group with tibial cortical defect. While healing within the defect was not altered by transient muscle paralysis vs saline in mice (in either extent or rapidity), almost complete inhibition of periosteal intramembraneous bone formation was observed. While not wishing to be bound by theory, it is believed that in uni-cortical bone defect healing (for defects of this size relative to the size of the tibia), the periosteal intramembraneous bone formation is not structurally required (i.e., required to stabilize the bone to bear load during healing), but is more likely a biologic pathway that is initiated as an unnecessary sequela (i.e., the pathway is in place, should it be required). In view of these data, transient muscle paralysis can prophylactically inhibit formation of bone in any physiologically unnecessary location (i.e., heterotopic ossification).

### Example 3

### Inhibition of Neuromuscular Function Proximal to Surgically-Induced Bone Trauma Inhibits the Periosteal Osteogenic Response to Skeletal Trauma but not Bone Formation at the Defect Site

In a separate control experiment, botulinum toxin A (BT×A; Botox®, Allergan; 2 U/100 g BW in 20 μl final volume) was injected into the quadriceps muscles of mice that underwent surgically induced skeletal trauma of the tibia. As the quadriceps muscle group overlies the femur, the purpose of this control was to determine if inhibition of neuromuscular function proximal to the defect site would alter the osteogenic response to skeletal trauma.

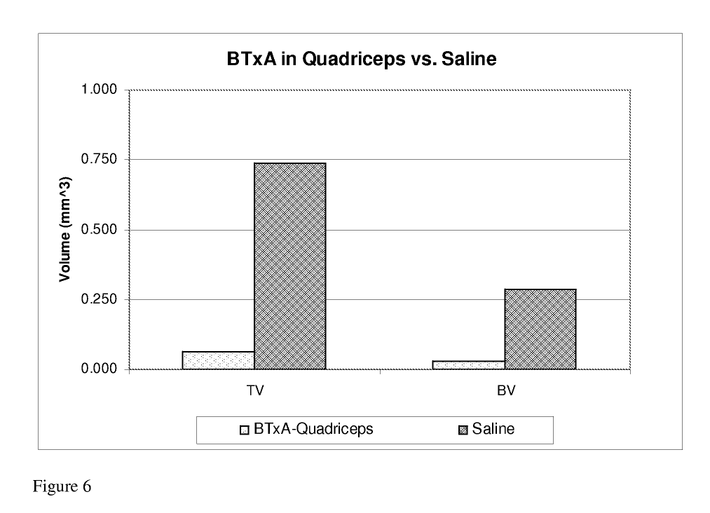

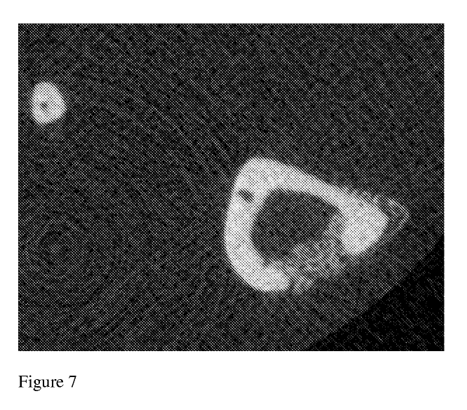

Compared to the saline injected controls, BT×A injection of the quadriceps led to nearly complete inhibition of the osteogenic response to skeletal trauma of the tibia; total volume of the callus was 8.8% and bone volume was 10.4% vs. controls (FIG. 6). Significantly, there was no effect of the muscle paralysis on bone formation at the defect site itself (FIG. 7).

### Example 4

### Demonstration that Blockade of Neuromuscular Signaling Prevents and/or Diminishes Heterotopic Ossification in a Widely Implemented Model of Ectopic Bone Formation

In these experiments, 16 wk old C57BL/6 mice are implanted with BMP4 impregnated Matrigel (BMP4-M) in the calf muscles to induce HO. The effect of neuromuscular blockade on the onset of HO is tested by injecting botulinum toxin A into the calf muscle group 24 hours prior to the BMP4-M implantation. Immediately following BMP4-M implantation, all mice undergo high-resolution microCT imaging of a 4 mm region of the tibia spanning the calf muscle implant site. A second scan is performed in all mice from each of the experimental groups one week after BMP4-M implantation and again at 2 and 3 weeks post-implantation. The primary outcome measure is mineralized nodule volume and BMD as determined by microCT scanning to determine the magnitude (i.e. volume) and maturation (i.e. BMD) of HO.

### Heterotopic Ossification Model

Heterotopic ossification is induced in the mid belly of the mouse right calf muscle by injection of growth factor-reduced Matrigel (Becton Dickinson) impregnated with BMP4 at a concentration of 2.5 μg in 50 μL [Glaser D L, Economides A N, Wang L, Liu X, Kimble R D, Fandl J P, Wilson J M, Stahl N, Kaplan F S, Shore E M 2003. In vivo somatic cell gene transfer of an engineered Noggin mutein prevents BMP4- induced heterotopic ossification. J Bone Joint Surg Am 85-A(12):2332-42]. The implants are prepared by adding the BMP4 to Matrigel at 4° C., when Matrigel is liquid. Upon injection into the muscle the mixture solidifies at ambient body temperature (i.e. 37° C.) and forms a stable nodule that undergoes ectopic mineralization. Based on pilot experiments in our laboratory, an injection volume of 50 μL induces a robust mineralizing HO nodule in the calf muscle within 21 days (FIG. 6).

### BT×A Muscle Paralysis Treatment

To assess whether motor and/or proprioceptive blockade inhibits the development of HO, study animals receive botulinum toxin A (BT×A; Botox®, Allergan; 2 U/100 g BW in 20 μL final volume) or saline injections into the right calf muscle (consisting of the gastrocnemius, soleus and plantaris muscles) 24 hours prior to the injection of the BMP4-Matrigel implant. At 24 hr post injection, calf muscle paralysis is confirmed in all mice by visual examination to assess whether the mice can extend their toes or demonstrate ankle plantar flexion during transient tail suspension.

Blockade of neuromuscular signaling will prevent or diminish heterotopic ossification in this accepted model of HO.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

1-11. (canceled)

12. A method of inhibiting heterotopic ossification (HO) in a subject in need thereof, said method comprising:
administering an effective amount of a proprioception inhibitor to the subject, whereby HO is inhibited or prevented.

13. The method according to claim 12, wherein said administering is carried out intramuscularly or by implantation.

14. The method according to claim 12, wherein said administering comprises administering said proprioception inhibitor to muscle adjacent to a transcortical bone defect.

15. The method according to claim 12, wherein the proprioception inhibitor is administered with a pharmaceutically-acceptable carrier.

16. The method according to claim 12, wherein the subject is a mammal.

17. The method according to claim 12, wherein the proprioception inhibitor is selected from the group consisting of inhibitors of small-diameter sensory fibers, saxitoxin, C. botulinum toxin, type A and other botulinum toxin preparations.

18. (canceled)

19. The method of claim 17, wherein the inhibitor of small-diameter sensory fibers is selected from the group consisting of lidocaine, bupivicaine, and veratridine.

20. The method according to claim 12, wherein the HO is due to or precipitated by an insult selected from the group consisting of spinal cord injury, traumatic brain injury, a burn, a fracture, muscle contusion, joint arthroplasty, amputation following trauma, a lower motor neuron disorder, and a hereditary disorder.

21. A method of treating a subject with bone trauma, said method comprising:
administering a proprioception inhibitor to the subject under conditions effective to treat the bone trauma, wherein the proprioception inhibitor inhibits or prevents HO.

22. The method according to claim 21, wherein said administering is carried out intramuscularly or by implantation.

23. The method according to claim 21, wherein said administering comprises administering said proprioception inhibitor to muscle adjacent to a transcortical bone defect.

24. The method according to claim 21, wherein the proprioception inhibitor is administered with a pharmaceutically-acceptable carrier.

25. The method according to claim 21, wherein the subject is a mammal.

26. The method according to claim 21, wherein the proprioception inhibitor is selected from the group consisting of inhibitors of small-diameter sensory fibers, saxitoxin, C. botulinum toxin, type A, and other botulinum toxin preparations.

27. (canceled)

28. The method of claim 26, wherein the inhibitor of small-diameter sensory fibers is selected from the group consisting of lidocaine, bupivicaine, and veratridine.

29-30. (canceled)
